(19) United States
(12) Patent Application Publication
(10) Pub. No.: US 2026/0085283 A1
(43) Pub. Date: Mar. 26, 2026

(54) STEM CELL-DERIVED GASTRULOID MODEL, METHOD FOR CONSTRUCTING SAME, AND USE

(71) Applicant: Innovation Center of Suzhou Nanjing Medical University, Jiangsu (CN)

(72) Inventors: Jiahao SHA, Nanjing (CN); Yan YUAN, Nanjing (CN); Gege YUAN, Nanjing (CN)

(21) Appl. No.: 19/111,159

(22) PCT Filed: Dec. 29, 2022
(86) PCT No.: PCT/CN2022/143274

§ 371 Date: Mar. 12, 2025

(30) Foreign Application Priority Data
Sep. 19, 2022 (CN) 202211135940.2

(51) Int. Cl. C12N 5/0735; G01N 33/50

(52) U.S. Cl. CPC C12N 5/0606; G01N 33/5091; C12N 2500/32; C12N 2500/44; C12N 2501/115; C12N 2501/155; C12N 2501/16; C12N 2501/727; C12N 2501/998; C12N 2503/02; C12N 2513/00

(57) ABSTRACT

Provided are a stem cell-derived gastruloid model, method for constructing same, and use thereof. The gastruloid model is constructed in vitro from human pluripotent stem cells, and simulated the biological events of early embryonic development and the key structures of the embryo to a certain extent, such as the development of primitive endoderm, the establishment of bilaminar germ disc, the appearance of the amniotic cavity and amniotic cells, and the appearance of the primitive streak and the like, which has been verified at both the protein level and the transcriptome level, and the key features of embryos from the peri-implantation period to the gastrula period could be well reproduced. This model can be induced in batches as a drug screening model for early embryos and environmental toxicants and the like, thereby providing safety testing for the use of drugs for certain clinical patients in early pregnancy and so on.

### CROSS-REFERENCE TO RELATED APPLICATIONS

This application is a U.S. National Stage of International Patent Application No. PCT/CN2022/143274 filed Dec. 29, 2022, which claims priority to Chinese Patent Application No. 202211135940.2 filed Sep. 19, 2022, both of which are incorporated by reference herein as if reproduced in their entireties.

### TECHNICAL FIELD

The present disclosure relates to the field of biotechnology, particularly to an in vitro simulated peri-implantation human gastruloid model derived from pluripotent stem cells, and an induction method and use thereof.

### BACKGROUND

Human life begins with fertilization, when a sperm and an egg combine to form a zygote. This unique totipotent cell undergoes continuous division and cell differentiation, forming a blastocyst on the fifth day. The blastocyst is mainly composed of two cell groups: the inner cell mass (ICM) and the trophectoderm (TE). The inner cell mass forms the embryo itself and extra-embryonic tissues in later development, while the trophectoderm develops further to form the main components of the placenta. Before embryo implantation, the inner cell mass begins to differentiate into the epiblast (EPI) and the hypoblast (HYPO). After implantation, the EPI forms the amniotic sac and the HYPO forms the yolk sac, and then the EPI undergoes gastrulation to establish a gastrula with three germ layers. Due to reasons such as technical and ethical restrictions and limited sample numbers, there are relatively few studies on human peri-implantation embryonic development. Understanding the mechanisms of early human embryonic development is very important for developmental biology and regenerative medicine. Currently, most of our understanding of early human embryonic development comes from the studies of the histological and anatomical structure of Carnegie embryos, and there are still many unknown areas to be explored at this stage.

At present, there has been a breakthrough in the study of human embryo development during the pre-implantation and peri-implantation periods, and researchers have been able to culture human embryos in vitro to the embryo before gastrulation at the 14th day, or induce naïve human pluripotent stem cells (nhPSCs) into pre-implantation embryoids. Combined with single-cell multi-omics sequencing and fluorescence imaging technology, it has opened up a new way to study human gastrulation development and greatly expanded the understanding of the characteristics and mechanisms of early human gastrulation development. Although some studies have attempted to use hPSCs to establish amniotic organoids or reconstruct the early development process of the primitive ectoderm to simulate the process of post-implantation embryonic development from a three-dimensional level, the model lacks the key structures of the embryo (bilaminar germ disc, yolk sac), and the general development process of the human primitive endoderm has not been observed. Therefore, an ideal research model of gastruloid from the peri-implantation period to the post-implantation period has not yet been established and obtained so far.

### SUMMARY

In this study, a model and a method for inducing human pluripotent stem cells (hPSCs) into gastruloids has been established. The gastruloid model is constructed in vitro and simulated the biological events of early embryonic development and the key structures of the embryo to a certain extent, such as the establishment of bilaminar germ disc, the appearance of the amniotic cavity and amniotic cells, and the appearance of the primitive streak and the like, which has been verified at both the protein level and the transcriptome level, and the key features of embryos from the peri-implantation period to the gastrula period could be well reproduced. Moreover, this model can be induced in batches as a drug screening model for early embryos, thereby providing safety testing for the use of drugs for certain clinical patients in early pregnancy.

The human pluripotent stem cells used in the present disclosure include human induced pluripotent stem cells (hiPSCs) and human embryonic stem cells (hESCs), the human induced pluripotent stem cells and the human embryonic stem cells are already established and can be purchased through public channels. In a particular embodiment, the human induced pluripotent stem cells are purchased from the Cell Bank/Stem Cell Bank of the Chinese Academy of Sciences Typical Culture Collection Committee, with the cell name DYR0100, and the catalog number SCSP-1301; the human embryonic stem cells are purchased from the Cell Bank/Stem Cell Bank of the Chinese Academy of Sciences Typical Culture Collection Committee, with the cell name H1, and the catalog number SCSP-301.

The first object of the present disclosure is to provide a method for constructing a gastruloid model, specifically, for inducing pluripotent stem cells into a human gastruloid model in vitro, comprising the following steps:
(1) digesting pluripotent stem cells into single cells when the cells grow to 80%-90% confluence, and resuspending the cells using PBS (phosphate buffered saline) to obtain a cell suspension 1; preferably, digesting the cells into single cells using trypsin TrypLE Select; taking the cell suspension 1 and adding a GIER culture medium containing Y-27632 to further resuspend the cells to obtain a cell suspension 2; inoculating the cell suspension 2 into an embryoid body culture plate coated with an anti-adhesion solution in advance, and centrifuging; replacing half of the culture medium with a GIER culture medium on the second day of culture, and replacing half of the culture medium every day until a primitive endoderm and a primitive ectoderm are formed; preferably, the ratio of the cell suspension 1 to the GIER culture medium containing Y-27632 is: adding 2 mL of the GIER culture medium containing Y-27632 to each cell suspension 1 containing 7.5×10⁴ cells to 9.0×10⁴ cells; preferably, the cell amount of the cell suspension 2 inoculated in each microwell is 50-75 cells; preferably, the centrifuging is performed using a horizontal centrifuge at 800 rpm for 2 minutes; preferably, the anti-adhesion solution is an Anti-adhesion rinsing Solution, and the embryoid body culture plate is an Agreewell 400™ culture dish; preferably, step (1) is performed on day 0 to day 3 of the method for constructing the post-implantation gastruloid model.
(2) Discarding ½ to ¾ of the volume of the G1ER culture medium in each well of the culture plate in step (1) and adding a G2EE culture medium for culturing, and performing amniotic cavity pre-induction; when adding the culture medium, the culture medium may be added slowly along the wall to prevent the formed gastruloids from floating up from the microwells and then fusing.

In a particular embodiment, the culture plate is an Agreewell 400™ culture dish, and ¾ of the volume of the G1ER culture medium in each well of the culture plate, i.e., 1.75 mL/well, is discarded. Preferably, step (2) is performed on day 4 of the method for constructing a post-implantation gastruloid model.
(3) Discarding ½ to ¾ of the volume of the G2EE medium in each well of the culture plate in step (2) and adding an aG2EE medium for culturing, and inducing the formation of amnion cells; when adding the culture medium, the culture medium may be added slowly along the wall to prevent the formed gastruloids from floating up from the microwells and then fusing.

In a particular embodiment, the culture plate is an Agreewell 400™ culture dish, and ¾ of the volume of the G2ER culture medium in each well of the culture plate, i.e., 1.75 mL/well, is discarded. Preferably, step (3) is performed on day 5 of the method for constructing a post-implantation gastruloid model.
(4) Discarding ½ to ¾ of the volume of the aG2EE culture medium in each well of the culture plate in step (3) and adding a bG2EE culture medium for culture; until the cells are induced into amnion cells, primitive streak cells, and primordial germ cells, the post-implantation gastruloid model is obtained. When adding the culture medium, the culture medium may be added slowly along the wall to prevent the formed gastruloids from floating up from the microwells and then fusing.

In a particular embodiment, the culture plate is an Agreewell 400™ culture dish, and ¾ of the volume of the aG2EE culture medium in each well of the culture plate, i.e., 1.75 mL/well, is discarded. Preferably, step (4) is performed on day 6 to day 7 of the method for constructing a post-implantation gastruloid model.

Furthermore, the culture conditions of the culture in steps (1) to (4) are: cultured in an environment with a temperature of 37° C. and a volume concentration of carbon dioxide of 5%-6%.

Furthermore, the pluripotent stem cells are induced pluripotent stem cells or embryonic stem cells; preferably, the pluripotent stem cells are human induced pluripotent stem cells or human embryonic stem cells.

Furthermore, the components of the GIER culture medium in step (1) comprise: 50% by volume of G-1™ Plus blastomere culture medium, 25% by volume of Essential 8 culture medium and 25% by volume of RACL culture medium; the RACL culture medium contains 95%-98.5% by volume of basic culture medium RPMI 1640 (containing GlutaMAX), 1% by volume of B27 additive, 0.5% by volume of Penicillin-Streptomycin, 0.1 mM non-essential amino acids, 0.1 mM β-mercaptoethanol, 100 ng/mL recombinant human activin A protein, 20 ng/mL recombinant human leukocyte factor protein, and 3 μM CHIR 99021.

The components of the GIER culture medium containing Y-27632 comprise: 50% by volume of G-1™ Plus blastomere culture medium, 25% by volume of Essential 8 culture medium and 25% by volume of RACL culture medium; the RACL culture medium contains 95%-98.5% of basic culture medium RPMI 1640 (containing GlutaMAX), 1% by volume of B27 additive, 0.5% by volume of Penicillin-Streptomycin 0.1 mM non-essential amino acids, 0.1 mM β-mercaptoethanol, 100 ng/mL recombinant human activin A protein, 20 ng/mL recombinant human leukocyte factor protein, 3 μM CHIR 99021, and 10 μM Y-27632.

Furthermore, the components of the G2EE culture medium in step (2) comprise: 50% by volume of G-2™ Plus embryo culture medium, 25% by volume of Essential 8 culture medium, and 25% by volume of EBB culture medium; the EBB culture medium contains 100% Essential 6 culture medium, 20 ng/mL recombinant human bone morphogenetic protein 4, and 10 ng/ml recombinant human fibroblast growth factor 2.

Furthermore, the components of the aG2EE culture medium in step (3) comprise: 50% by volume of G-2™ Plus embryo culture medium, 25% by volume of Essential 8 culture medium, and 25% by volume of aEBB culture medium; the aEBB culture medium contains 100% Essential 6 culture medium, 100 ng/ml recombinant human bone morphogenetic protein 4, and 10 ng/ml recombinant human fibroblast growth factor 2.

Furthermore, the components of the bG2EE culture medium in step (4) comprise: 50% by volume of G-2™ Plus embryo culture medium, 25% by volume of Essential 8 culture medium, and 25% by volume of bEBB culture medium; the bEBB culture medium contains 100% Essential 6 culture medium, 75 ng/ml recombinant human bone morphogenetic protein 4, and 10 ng/ml recombinant human fibroblast growth factor 2.

The second object of the present disclosure is to provide a gastruloid model constructed by the aforementioned method.

The third object of the present disclosure is to provide the use of the aforementioned gastruloid model, or tissues or organs derived from the model, or cultures thereof in the study of the mechanism of human early embryonic development.

The fourth object of the present disclosure is to provide the use of the aforementioned gastruloid model, or tissues or organs derived from the model, or cultures thereof in diagnostic strategies and/or therapeutic strategies for diseases of human early embryonic development.

The fifth object of the present disclosure is to provide the use of the aforementioned gastruloid model, or tissues or organs derived from the model, or cultures thereof in the screening, verification, evaluation, assessment or study of the efficacy of the medicament for preventing and/or treating diseases of human early embryonic development.

Further, the diseases of human early embryonic development are human early embryo teratogenesis caused by drugs.

The gastruloid model described in the present disclosure is a model with a final state similar to the Carnegie stage 6 embryo, obtained by inducing pluripotent stem cells in vitro. This model can be induced in batches as a drug screening model for early embryos, thereby providing safety testing for the use of drugs for certain clinical patients in early pregnancy. The gastruloid model, or the tissue or the organ derived from the gastruloid model, or the culture thereof cannot develop into an individual due to the absence of cell types such as trophoblast.

The technical solution of the present disclosure has the following advantages.
Feature 1: Human pluripotent stem cells can aggregate into spheroids within 12 hours, have high induced stability, and have stable cell growth characteristics in continuous 7-day gastruloid culture.
Feature 2: On day 1 of gastruloid induction, lineage separation of primitive endoderm and primitive ectoderm begins, and primitive endoderm-like cells appears, expressing markers of pluripotency (OCT4) and early primitive endoderm (OTX2, GATA6) simultaneously.
Feature 3: On day 3, the typical bilaminar germ disc structure of embryo appears, the cells on the Epiblast side are OCT4 and SOX2 positive columnar cells, and the cells on the Hypoblast side are OTX2, GATA6, and SOX17 positive cuboidal cells.
Feature 4: On days 4 and 5 of gastruloid induction, the amniotic cavity structure begins to form on the Epiblast side, and some amniotic cells appears, showing TFAP2a and CDX2 positive, OCT4 weakly positive, and SOX2 negative.
Feature 5: On days 6 and 7 of gastruloid induction, the amniotic cells separated from Epiblasts further thinned to form a distinct squamous amniotic epithelium, showing positive amniotic cell markers such as TFAP2a and CDX2 and negative pluripotency markers such as OCT4 and SOX2.
Feature 6: On days 6 and 7 of gastruloid induction, some T and EOMES-positive cells are separated from Epiblast, and the pluripotency marker (OCT4) is further weakened, the epithelial-mesenchymal transition (EMT) process is in progress, which is consistent with the characteristics of primitive streak generation, proving that gastrulation begins to initiate in post-implantation gastruloid.
Feature 7: On days 6 and 7 of gastruloid induction, a small number of primordial germ cells appear, showing pluripotency markers (OCT4, NANOG, NANOS3) positive, primordial germ cells (SOX17, AP2C, BLIMP1) positive, and characteristic SOX2 negative.
Feature 8: The final state of the gastruloid after 7 days of induction culture is similar to that of the embryo at Carnegie stage 6.
Feature 9: This model can be induced in batches as a drug screening model for early embryos, thereby providing safety testing for the use of drugs for certain clinical patients in early pregnancy.

Beneficial effects: This model provides a platform for in vitro studies on early human embryonic development, aims to understand the complexity of early human embryonic development, and provides a research platform for the development of clinical treatments for early embryonic peri-implantation diseases.

### DESCRIPTION OF THE DRAWINGS

FIG. 1 is a white light image of the culture process of the gastruloid model provided by an example of the present disclosure induced for 1-7 days, wherein A is a post-implantation gastruloid induced from human induced pluripotent stem cells (hiPSCs), and B is a post-implantation gastruloid induced from human embryonic stem cells (hESCs), with a scale bar of 100 μm.

FIG. 2 is a white light image of the sampling process of the post-implantation gastruloid induced from human induced pluripotent stem cells (hiPSCs) at 3 days and 7 days of induction, with scale bars of 50 μm and 100 μm.

FIG. 3 is a growth curve of gastruloid provided by an example of the present disclosure at 1-7 days of induction, each point represents the amount of cells in a blastoid, and the number of samples at each time point is not less than 20, wherein A is a growth curve at 1-7 days of induction from human induced pluripotent stem cells (hiPSCs), and B is a growth curve at 1-7 days of induction from human embryonic stem cells (hESCs).

FIG. 4 is immunofluorescence (IF) images of the process from the appearance of primitive endoderm to the appearance of bilaminar germ disc structure during the 1-3 days of the induction of the gastruloid provided by an example of the present disclosure, with a scale bar of 20 μm; wherein A is an immunofluorescence image of GATA6 and OCT4, and B is an immunofluorescence image of SOX2 and SOX17.

FIG. 5 is immunofluorescence images of the process from the formation of the amniotic cavity to the appearance of amniotic cells during the 4-7 days of induction of the gastruloid provided by an example of the present disclosure, with a scale bar of 20 μm; wherein A is CDX2, OCT4 and GATA6, and B is SOX2, TFAP2a and SOX17.

FIG. 6 is immunofluorescence images of the primitive streak migration process of gastrulation at 7 days of induction of the gastruloid provided by an example of the present disclosure, wherein A shows that OCT4+T+ gastrulation cells gradually transition to T+EOMES+positive cells with definitive endoderm fate, and finally differentiate into EOMES+ definitive endoderm cells, and B shows that OCT4+T+ gastrulation cells gradually transition to T+MIXL1+ positive cells with mesoderm fate, and finally differentiate into MIXL1+ mesoderm cells, with a scale bar of 50 μm.

FIG. 7 is a bioinformatics analysis result provided by an example of the present disclosure, A is a UMAP clustering diagram of single-cell sequencing data at 7 days of induction of the gastruloid, wherein Epiblast is the primitive ectoderm, Endoderm is the primitive endoderm, Primitive Streak is the primitive streak, and Primitive Streak anlage Epiblast is the primitive streak primordium ectoderm, PGC is the primordial germ cell, Amnion is the amnion, and Mesoderm is the mesoderm; B is the relevant basis for grouping.

FIG. 8 is the result of dosing application of the gastruloid model provided by an example of the present disclosure, A is the white light image of sampling of the gastruloids of the control group, dimethyl sulfoxide, 5 μM THD and 10 μM THD at day 7 and the DAPI (4′,6-diamidino-2-phenylindole) staining result, with scale bars of 200 μm and 100 μm, respectively; B is the cell amount of a single gastruloid in the control group, dimethyl sulfoxide, 5 μM THD and 10 μM THD at 7 days of induction of the gastruloid; C is the volume of a single gastruloid of the control group, dimethyl sulfoxide, 5 μM THD and 10 μM THD at 7 days of induction of the gastruloid.

### DETAILED DESCRIPTION

The present disclosure discloses a method for inducing human pluripotent stem cells into a gastruloid model, and the reagents, instruments, cell lines and the like used in the method for inducing human pluripotent stem cells into a gastruloid model provided by the present disclosure can all be purchased from the market, the human induced pluripotent stem cells are purchased from the Cell Bank/Stem Cell Bank of the Chinese Academy of Sciences Typical Culture Collection Committee, with the cell name DYR0100, and the catalog number SCSP-1301; the human embryonic stem cells are purchased from the Cell Bank/Stem Cell Bank of the Chinese Academy of Sciences Typical Culture Collection Committee, with the cell name H1, and the catalog number SCSP-301.
(1) The sources of the components of the culture medium used for inducing human pluripotent stem cells into gastruloid in vitro are as follows: G-1™ Plus blastomere culture medium: 10128, purchased from Vitrolife, Sweden; G-2™ Plus embryo culture medium: 10132, purchased from Vitrolife, Sweden; Essential 8 culture medium: A1517001, purchased from Gibco, USA; Essential 6 culture medium: A1516401, purchased from Gibco, USA; RPMI 1640 (containing GlutaMAX): 61870036, purchased from Gibco, USA; Anti-adhesion rinsing solution: 07010, purchased from STEMCELL, USA; B27 additive: 12587-010, purchased from Gibco, USA; non-essential amino acids, Gibco® MEM non-essential amino acids, 11140076, purchased from Gibco, USA; β-mercaptoethanol: 21985023, purchased from Gibco, USA; recombinant human bone morphogenetic protein 4:314-BP, purchased from R&D Systems, USA; recombinant human leukemia inhibitory factor: 225-SC, purchased from R&D Systems, USA; recombinant human activin A protein: 338-AC, purchased from R&D Systems, USA; recombinant human fibroblast growth factor 2:3718-FB, purchased from R&D Systems, USA; ROCK inhibitor: specific types are Y27632 and HY-10071, purchased from MCE, USA; CHIR99021: specific types are Laduviglusib trihydrochloride and HY-10182B, purchased from MCE, USA.
(2) Preparation of the G1ER culture medium: 50% by volume of G-1™ Plus blastomere culture medium, 25% by volume of Essential 8 culture medium and 25% by volume of RACL culture medium; the RACL culture medium contains 98.5% by volume of basic culture medium RPMI 1640 (containing GlutaMAX), 1% by volume of B27 additive, 0.5% by volume of Penicillin-Streptomycin, 0.1 mM non-essential amino acids, 0.1 mM β-mercaptoethanol, 100 ng/ml recombinant human activin A protein, 20 ng/mL recombinant human leukocyte factor protein, and 3 μM CHIR 99021.
(3) Preparation of the GIER culture medium containing Y-27632: 50% by volume of G-1™ Plus blastomere culture medium, 25% by volume of Essential 8 culture medium, and 25% by volume of RACL culture medium; the RACL culture medium contains 98.5% of basic culture medium RPMI 1640 (containing GlutaMAX), 1% by volume of B27 additive, 0.5% by volume of Penicillin-Streptomycin, 0.1 mM non-essential amino acids, 0.1 mM β-mercaptoethanol, 100 ng/ml recombinant human activin A protein, 20 ng/mL recombinant human leukocyte factor protein, 3 μM CHIR 99021, and 10 μM Y-27632.
(4) Preparation of the G2EE culture medium: 50% by volume of G-2™ Plus embryo culture medium, 25% by volume of Essential 8 culture medium, and 25% by volume of EBB culture medium; the EBB culture medium contains 100% Essential 6 culture medium, 20 ng/ml recombinant human bone morphogenetic protein 4, and 10 ng/mL recombinant human fibroblast growth factor 2.
(5) Preparation of the aG2EE culture medium: 50% by volume of G-2™ Plus embryo culture medium, 25% by volume of Essential 8 culture medium, and 25% by volume of aEBB culture medium; the aEBB culture medium contains 100% Essential 6 culture medium and multiple cytokines: 100 ng/mL recombinant human bone morphogenetic protein 4, and 10 ng/ml recombinant human fibroblast growth factor 2.
(6) Preparation of the bG2EE culture medium: 50% by volume of G-2™ Plus embryo culture medium, 25% by volume of Essential 8 culture medium, and 25% by volume of bEBB culture medium; the bEBB culture medium contains 100% Essential 6 culture medium and multiple cytokines: 75 ng/ml recombinant human bone morphogenetic protein 4, and 10 ng/ml recombinant human fibroblast growth factor 2.

### Example 1 Induction of Gastruloids from Human Induced Pluripotent Stem Cells (iPSCs) (First Stage): Days 0-3, Formation of Primitive Endoderm and Primitive Ectoderm

When human induced pluripotent stem cells DYR0100 grew to 80-90% confluence, TrypLE Select was used to digest the cells into single cells, and the cells were resuspended in PBS to obtain a cell suspension 1; Cell suspension 1 containing 9.0×10+ cells was taken, and 2 mL GIER culture medium containing Y-27632 was added to resuspend the cells to obtain a cell suspension 2.

The cell suspension 2 was inoculated into a well of an Agreewell 400™ culture dish coated with the anti-adhesion solution (anti-adhesion rinsing solution) in advance; after centrifugation at 800 rpm for 2 minutes using a horizontal centrifuge, the cell amount per microwell was 75 cells (each well of the Agreewell 400™ culture dish has 1200 microwells, that is, the cell amount in each microwell was 9.0×10⁴/1200=75 cells).

On day 2 of the culture, half of the medium was replaced with the GIER medium every day until day 3 of the culture to form the primitive endoderm and the primitive ectoderm.

### Example 2 Induction of Gastruloid from Human Induced Pluripotent Stem Cells (hiPSCs) (Second Stage): Day 4, Pre-Induction of Amniotic Cavity

After discarding ¾ volume/well (i.e. 1.75 mL/well) of the first-stage GIER culture medium, the second-stage G2EE culture medium was added slowly along the wall to prevent the formed gastruloids from floating up from the microwells and then fusing, and the pre-induction of amniotic cavity was performed.

### Example 3 Induction of Gastruloid from Human Induced Pluripotent Stem Cells (hiPSCs) (Third Stage): Day 5, Induction of Amniotic Cells

After discarding ¾ volume/well (i.e. 1.75 mL/well) of the second-stage G2EE culture medium, the third-stage aG2EE culture medium was added slowly along the wall to prevent the formed gastruloids from floating up from the microwells and then fusing, and the formation of amnion cells was induced.

### Example 4 Induction of Gastruloid from Human Induced Pluripotent Stem Cells (hiPSCs) (Fourth Stage): Days 6-7, Induction of Amniotic Cells, Primitive Streak Cells, and Primordial Germ Cells

After discarding ¾ volume/well (i.e. 1.75 mL/well) of the third-stage aG2EE culture medium, the fourth-stage bG2EE culture medium was added slowly along the wall to prevent the formed gastruloids from floating up from the microwells and then fusing. After 24 hours, the medium was continued to replace by 1.75 mL (the bG2EE culture medium), and cultured until day 7 to induce into amniotic cells, primitive streak cells, and primordial germ cells, and the post-implantation gastruloid model was obtained.

### Example 5 Induction of Gastruloid from Human Embryonic Stem Cells (hESCs), Repeating the Experimental Contents of Examples 1-4

(1) Days 0-3, formation of primitive endoderm and primitive ectoderm. When human embryonic stem cells H1 grew to 80-90% confluence, TrypLE Select was used to digest the cells into single cells, and the cells were resuspended in PBS to obtain a cell suspension 1; Cell suspension 1 containing 9.0×10+ cells was taken, and 2 mL GIER culture medium containing Y-27632 was added to resuspend the cells to obtain a cell suspension 2.

The cell suspension 2 was inoculated into a well of an Agreewell 400™ culture dish coated with the anti-adhesion solution (Anti-adhesion rinsing Solution) in advance, after centrifugation at 800 rpm for 2 minutes using a horizontal centrifuge, the number of cells per microwell was 75 cells (each well of the Agreewell 400™ culture dish has 1200 microwells, that is, the cell amount in each microwell was 9.0×10⁴/1200=75 cells).

On day 2 of the culture, half of the culture medium was replaced with the GIER culture medium every day until day 3 of the culture to form the primitive endoderm and primitive ectoderm.
(2) Day 4, pre-induction of amniotic cavity. After discarding ¾ volume/well (i.e. 1.75 mL/well) of the first-stage GIER culture medium, the second-stage G2EE culture medium was added slowly along the wall to prevent the formed gastruloids from floating up from the microwells and then fusing, and the pre-induction of amniotic cavity was performed.
(3) Day 5, pre-induction of amniotic cells. After discarding ¾ volume/well (i.e. 1.75 mL/well) of the second-stage G2EE culture medium, the third-stage aG2EE culture medium was added slowly along the wall to prevent the formed gastruloids from floating up from the microwells and then fusing, and the formation of amnion cells was induced.
(4) Days 6-7, induction of amniotic cells, primitive streak cells, and primordial germ cells. After discarding ¾ volume/well (i.e. 1.75 mL/well) of the third-stage aG2EE culture medium, the fourth-stage bG2EE culture medium was added slowly along the wall to prevent the formed gastruloids from floating up from the microwells and then fusing. After 24 hours, the culture medium was continuously replaced by 1.75 mL (the bG2EE culture medium), and cultured until day 7 to induce into amniotic cells, primitive streak cells, and primordial germ cells, and the post-implantation gastruloid model was obtained.

### Example 6 Verification of the Biological Characteristics of Gastruloids Induced from Human Induced Pluripotent Stem Cells (hiPSCs)

### 1.1 Morphological Observation of Induced Gastruloids

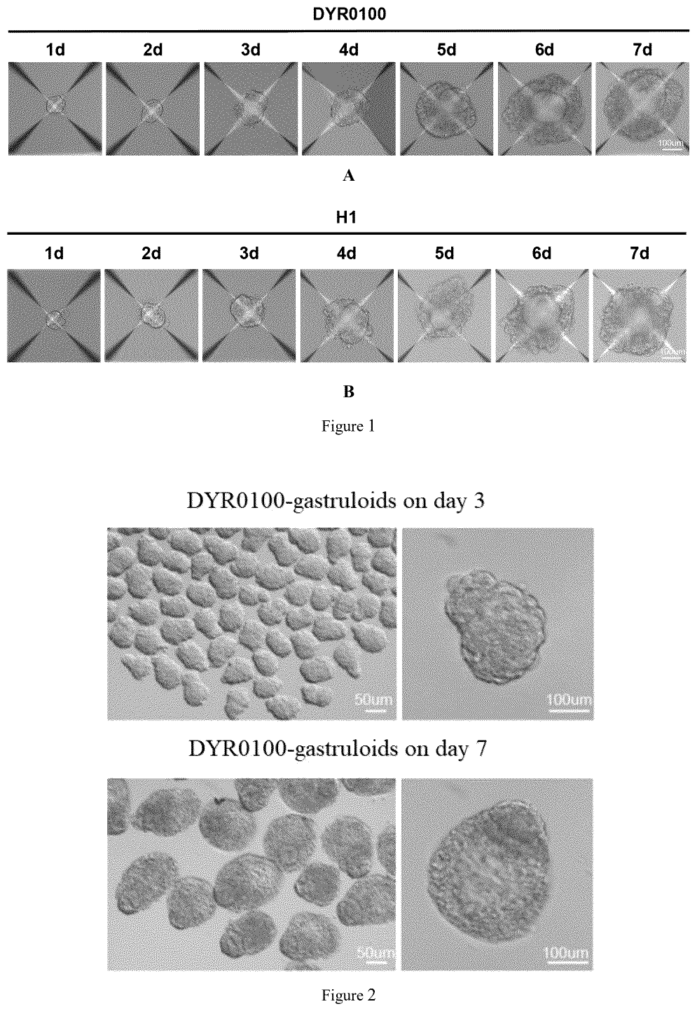

The gastruloid models induced for 1-7 days in the culture dishes in Examples 1 to 4 were observed under an upright fluorescence microscope. It can be seen that the cells grew rapidly, the volume of the gastruloids gradually increased, and there was an obvious bilaminar germ disc arrangement structure on day 3, an amniotic cavity structure appeared on days 4-5, and a yolk sac structure appeared in some gastruloids on days 4-7. The diameter of the gastruloids on day 7 ranged from about 250 μm to 350 μm. The morphological observation results are shown in A of FIG. 1 and FIG. 2.

### 1.2 Determination of the Growth Curve of Induced Post-Implantation Gastruloids

1.2.1 Growth curve determination steps. In the process of induction for 1-7 days in the culture dish in Examples 1 to 4, samples were taken every 24 hours. After sampling, 4% paraformaldehyde was used for fixation for 30 minutes, and the fixative was discarded and PBS was added for washing 3 times, 5 minutes/time. Then 5% bovine serum albumin (containing 1% tTriton) was used for blocking at room temperature for 4 hours. Then DAPI was diluted at a ratio of 1:200 and stained at room temperature for 4 hours. After discarding the DAPI dilution solution, PBS was added for washing 3 times, 5 minutes/time, and the sample was moved into a Chamber (staining chamber), and a certain amount of the prepared iohexol solution was added for transparency, and the Chamber was lightly covered with a slide and then photographed using a confocal microscope. After photographing, IMARIS software was used to adjust the parameters for single sample counting, and there were 21-36 replicates for each day's samples.

1.2.2 Results of growth curve determination. The growth curve data shown in Table 1 below were obtained after continuous measurement for 7 days. Table 1 shows the growth curve data obtained after continuous measurement for 7 days, with 21-36 replicates for each day's samples.

TABLE 1
Growth Curve Data

| | 1 d | 2 d | 3 d | 4 d | 5 d | 6 d | 7 d |
|---|---|---|---|---|---|---|---|
| 1 | 41 | 98 | 200 | 536 | 506 | 947 | 965 |
| 2 | 25 | 127 | 197 | 227 | 446 | 384 | 1082 |
| 3 | 45 | 101 | 201 | 422 | 494 | 845 | 700 |
| 4 | 31 | 117 | 362 | 473 | 647 | 735 | 972 |
| 5 | 50 | 132 | 168 | 212 | 621 | 1121 | 794 |
| 6 | 67 | 163 | 164 | 286 | 516 | 798 | 1122 |
| 7 | 58 | 119 | 170 | 405 | 523 | 404 | 899 |
| 8 | 50 | 116 | 268 | 544 | 514 | 1319 | 1103 |
| 9 | 53 | 129 | 186 | 296 | 542 | 933 | 867 |
| 10 | 48 | 137 | 209 | 274 | 484 | 949 | 965 |
| 11 | 42 | 88 | 237 | 401 | 429 | 888 | 1224 |
| 12 | 49 | 89 | 157 | 504 | 485 | 969 | 1348 |
| 13 | 66 | 93 | 202 | 388 | 702 | 1064 | 706 |
| 14 | 38 | 115 | 218 | 588 | 491 | 1161 | 1050 |
| 15 | 41 | 93 | 339 | 353 | 303 | 865 | 1232 |
| 16 | 34 | 175 | 212 | 365 | 436 | 1128 | 706 |
| 17 | 46 | 133 | 213 | 387 | 673 | 1164 | 1050 |
| 18 | 46 | 136 | 75 | 324 | 514 | 1222 | 1232 |
| 19 | 43 | 118 | 253 | 501 | 616 | 693 | 853 |
| 20 | 65 | 106 | 158 | 252 | 605 | 429 | 920 |
| 21 | 51 | 150 | 170 | 209 | 833 | 709 | 1201 |
| 22 | 46 | 117 | 230 | 528 | 729 | — | 1310 |
| 23 | 32 | 111 | 182 | 387 | 410 | — | 1006 |
| 24 | 38 | 101 | 216 | 405 | 604 | — | 1562 |
| 25 | 37 | 148 | 234 | 473 | 523 | — | 964 |
| 26 | 55 | 116 | 204 | 390 | 523 | — | 830 |
| 27 | 45 | 125 | 263 | 291 | 605 | — | — |
| 28 | 39 | — | 286 | 429 | 710 | — | — |
| 29 | 44 | — | 221 | 359 | — | — | — |
| 30 | — | — | 269 | 384 | — | — | — |
| 31 | — | — | 251 | 556 | — | — | — |
| 32 | — | — | 242 | 418 | — | — | — |
| 33 | — | — | 169 | — | — | — | — |
| 34 | — | — | 287 | — | — | — | — |
| 35 | — | — | 242 | — | — | — | — |
| 36 | — | — | 291 | — | — | — | — |

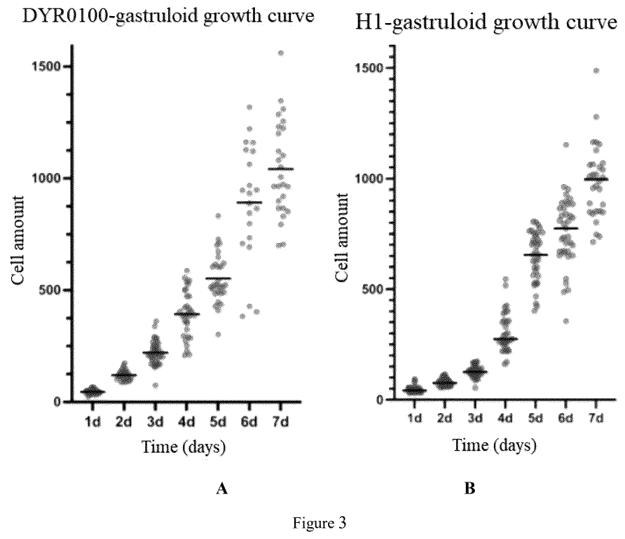

Based on the cell growth curve data in Table 1, a schematic diagram of the growth curve of the induced post-implantation gastruloids is obtained as shown in A of FIG. 3. The horizontal axis of FIG. 3 is the induction culture time (days), and the vertical axis is the number of cells in a single gastruloid.

Referring to the growth curve data in Table 1 and the growth curve schematic diagram shown in A of FIG. 3, it can be seen that the cells in gastruloid proliferated steadily, from an initial cell amount of 50-75 cells on day 0 to a cell amount of 1040 cells on day 7.

In summary, the cells of the induced gastruloid proliferate rapidly, have active cell growth, good cell activity, high cell induction stability, and have stable cell growth characteristics for in vitro induction.

### 1.3 Immunofluorescence (IF) Identification

1.3.1 Process of immunofluorescence identification. In the process of induction for 1-7 days in the culture dish in Examples 1 to 4, samples were taken every 24 hours. 4% paraformaldehyde was used for fixation for 30 minutes, and the fixative was discarded and PBST was added for washing 3 times, 5 minutes/time. 5% bovine serum albumin (containing 1% tTriton) was used for blocking at room temperature for 4 hours. The blocking solution was discarded, the antibody diluted in proportion was added, and incubated at 4° C. for 24 hours.

After discarding the primary antibody, PBS (containing 1% Triton) was added for washing 3 times, 5 minutes/time, the diluted secondary antibody and DAPI were added, and incubated at room temperature for 4 hours.

After discarding the secondary antibody, PBS (containing 1% Triton) was added for washing 3 times, 5 minutes/time, the sample was moved into the Chamber (staining chamber), and a certain amount of the prepared iohexol solution was added for transparency, and the Chamber was lightly covered with a slide and then photographed using a confocal microscope.

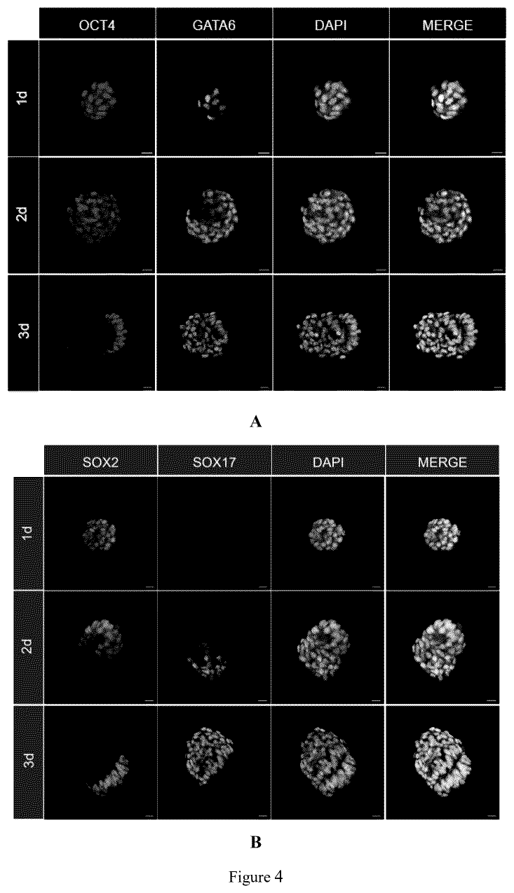

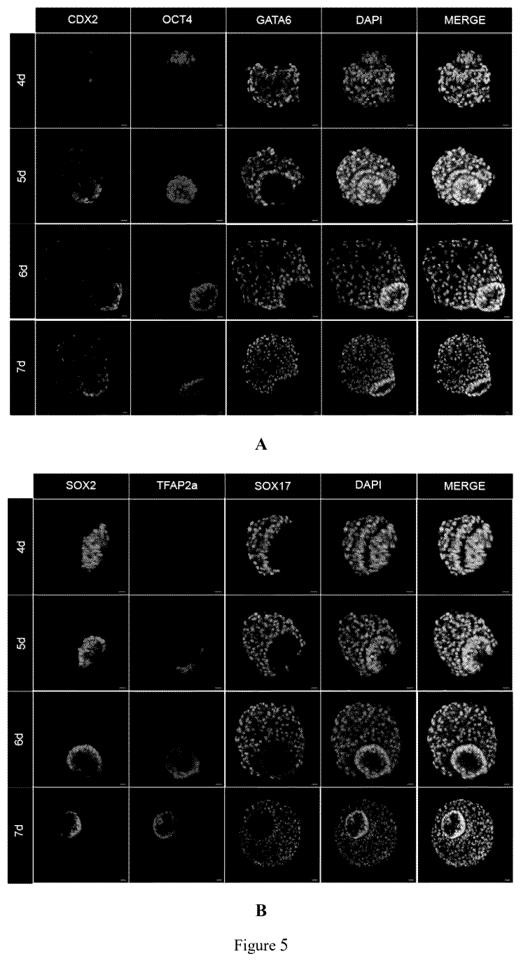

1.3.2 Results of immunofluorescence identification. The results of immunofluorescence identification are shown in FIGS. 4-6. On day 1 of induction, a small number of GATA6 and OCT4 double-positive cells appeared, mixed in the primitive ectoderm positive for OCT4 and SOX2, indicating that the lineage of the primitive endoderm began to differentiate. On day 2 of induction, the number of GATA6-positive cells began to increase, SOX17-positive cells appeared at the same time, and these cells gradually separated from the primitive ectoderm, further showing an increase in primitive endoderm cells. On day 3 of induction, it can be seen that the primitive endoderm cells positive for GATA6 and SOX17 were completely separated from the primitive ectoderm cells positive for OCT4 and SOX2, and a neatly arranged bilaminar germ disc structure was formed between the two cell types, in which the primitive ectoderm cell nuclei appeared as columnar stereoscopic cells, and the primitive endoderm cell nuclei appeared as dwarf columnar cells (FIG. 4).

On days 4-5 of induction, a cavity-like structure gradually appeared on the side of the primitive ectoderm, which was consistent with the characteristics of the amniotic cavity, and cells that were positive for transcription factor 2a (TFAP2a), CDX2 and OCT4, and negative for SOX2 appeared, becoming early amniotic cells. On days 6-7 of induction, mature amniotic cells that were positive for TFAP2a, CDX2, and negative for OCT4 and SOX2 appeared, and the morphology of the cell nucleus became flat, similar to mature squamous amniotic epithelial cells (FIG. 5).

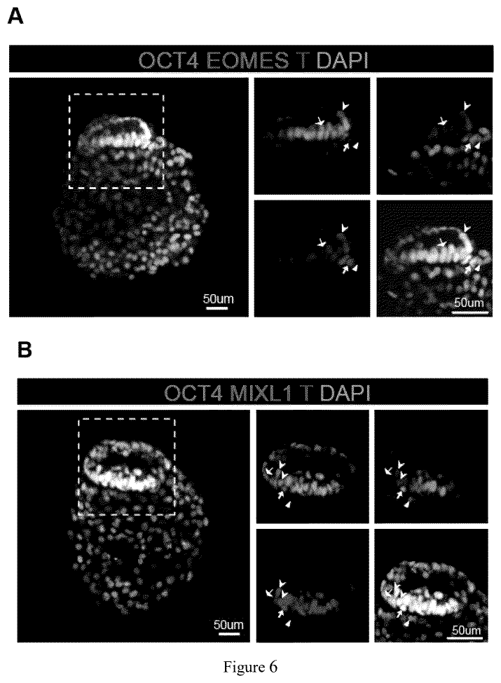

On days 6-7 of induction, T, EOMES, and MIXL1-positive cells appeared in the OCT4-positive columnar primitive ectoderm, and the OCT4 positivity gradually weakened, similar to the primitive streak cells, proving that the gastruloid began to undergo epithelial-mesenchymal transition (EMT) and began gastrulation (FIG. 6).

### 1.4 RNA Sequencing Identification

1.4.1RNA sequencing identification steps. The gastruloids on day 7 of induction in the culture dish in Examples 1 to 4 were sampled. The culture medium was removed, and the gastruloids were washed at least twice with PBS (0.01M, pH7.4) to remove the old culture medium and the detached dead cell debris, and digested with 2 mL of 0.24% Trypsin, placed in a 37° C. environment for digestion for 3 minutes, and a pipette was used to gently blow the gastruloids into single cells and then the digestion was terminated with a culture medium containing serum.

The culture medium was centrifuged at 1000 rpm for 5 minutes and the supernatant was removed. After resuspending in PBS, the solution was transferred to an enzyme-free EP tube for testing.

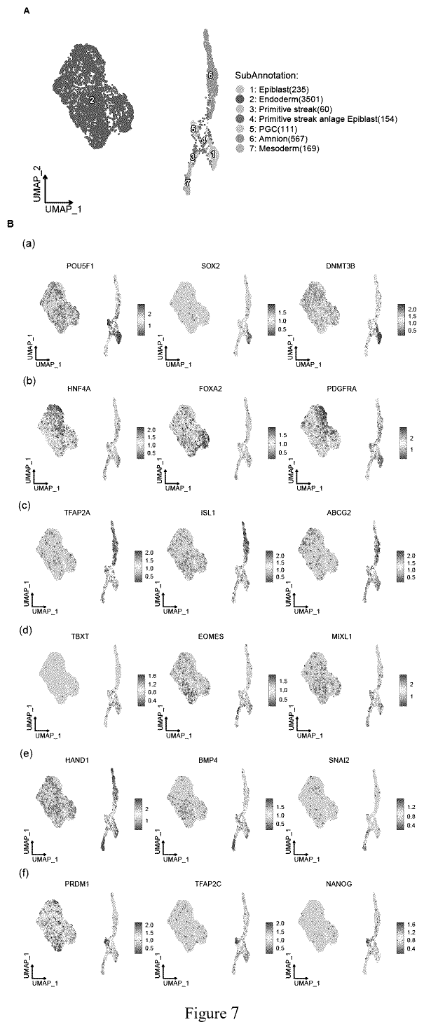

1.4.2 Results of RNA sequencing identification. The data of gastruloids on day 7 of induction were analyzed and clustered, the results showed that the cultured gastruloids were divided into 7 major groups, namely primitive ectoderm, primitive endoderm, primitive streak primordium cells, primitive streak, primordial germ cells, amnion and mesoderm, and the co-localization results of 3 known related specific markers were used for verification. Wherein, POIU5F1, SOX2, and DNMT3B were used to define the primitive ectoderm; HNF4A, FOXA2, and PDGFRA were used to define the primitive endoderm; TFAP2A, ISL1, and ABCG2 were used to define the amnion; TBXT, EOMES, and MIXL1 were used to define the primitive streak precursor cells and the primitive streak; HAND1, BMP4, and SNAI2 were used to define the mesoderm-related cells, and PRDM1, TFAP2C, and NANOG were used to define the primordial germ cells (FIG. 7).

### Example 7—Verification of the Biological Characteristics of Gastruloids Induced from Human Embryonic Stem Cells (hESCs)

### 1.1 Morphological Observation of Induced Post-Implantation Gastruloids

The gastruloid models induced for 1-7 days in the culture dishes in Example 5 were observed under an upright fluorescence microscope. It can be seen that the cells grew rapidly, the volume of the gastruloids gradually increased, and there was an obvious bilaminar germ disc arrangement structure on day 3, an amniotic cavity structure appeared on days 4-5, and a yolk sac structure appeared in some gastruloids on days 4-7. The diameter of the gastruloids on day 7 ranged from about 250 μm to 350 μm. The morphological observation results are shown in B of FIG. 1.

### 1.2 Determination of the Growth Curve of Induced Post-Implantation Gastruloids

1.2.1 Growth curve determination steps. During the 1-7 days of induction in the culture dish in Examples 5, samples were taken every 24 hours. After sampling, 4% paraformaldehyde was used for fixation for 30 minutes, and the fixative was discarded and PBS was added for washing 3 times, 5 minutes/time. 5% bovine serum albumin (containing 1% Triton) was used for blocking at room temperature for 4 h. Then DAPI was diluted at a ratio of 1:200 and stained at room temperature for 4 hours.

After discarding the DAPI dilution solution, PBS was added for washing 3 times, 5 minutes/time, and the sample was moved into the Chamber (staining chamber), and a certain amount of the prepared iohexol solution was added for transparency, and the Chamber was lightly covered with a slide and then photographed using a confocal microscope.

After photographing, IMARIS software was used to set and adjust the parameters for single sample counting, and there were 30-37 replicates for each day's samples.

1.2.2 Results of growth curve determination. The growth curve data shown in Table 1 below were obtained after continuous measurement for 7 days. Table 2 shows the growth curve data obtained after continuous measurement for 7 days, with 30-37 replicates for each day's samples.

TABLE 2
Growth Curve Data

| | 1 d | 2 d | 3 d | 4 d | 5 d | 6 d | 7 d |
|---|---|---|---|---|---|---|---|
| 1 | 41 | 80 | 54 | 223 | 763 | 811 | 1165 |
| 2 | 53 | 89 | 109 | 257 | 777 | 885 | 852 |
| 3 | 30 | 75 | 165 | 171 | 655 | 865 | 849 |
| 4 | 41 | 98 | 101 | 269 | 756 | 928 | 953 |
| 5 | 30 | 63 | 117 | 252 | 626 | 726 | 802 |
| 6 | 36 | 61 | 117 | 301 | 707 | 496 | 1018 |
| 7 | 42 | 85 | 135 | 228 | 527 | 952 | 989 |
| 8 | 37 | 83 | 174 | 330 | 754 | 356 | 967 |
| 9 | 41 | 84 | 142 | 390 | 739 | 647 | 1489 |
| 10 | 33 | 67 | 116 | 219 | 517 | 664 | 1280 |
| 11 | 42 | 86 | 129 | 397 | 672 | 487 | 852 |
| 12 | 40 | 100 | 158 | 275 | 750 | 775 | 747 |
| 13 | 39 | 70 | 125 | 401 | 721 | 1153 | 1004 |
| 14 | 55 | 65 | 95 | 268 | 791 | 796 | 847 |
| 15 | 94 | 67 | 88 | 250 | 806 | 526 | 1127 |
| 16 | 45 | 112 | 142 | 220 | 468 | 732 | 1165 |
| 17 | 39 | 57 | 131 | 219 | 402 | 652 | 1018 |
| 18 | 57 | 91 | 123 | 260 | 764 | 823 | 1065 |
| 19 | 39 | 94 | 169 | 290 | 722 | 652 | 1010 |
| 20 | 47 | 107 | 135 | 359 | 697 | 839 | 738 |
| 21 | 51 | 61 | 106 | 295 | 434 | 895 | 889 |
| 22 | 55 | 78 | 123 | 419 | 596 | 885 | 714 |
| 23 | 44 | 55 | 132 | 265 | 599 | 963 | 1051 |
| 24 | 46 | 63 | 122 | 161 | 524 | 708 | 840 |
| 25 | 48 | 71 | 119 | 516 | 655 | 737 | 1039 |
| 26 | 33 | 58 | 138 | 357 | 804 | 699 | 1071 |
| 27 | 53 | 104 | 156 | 427 | 627 | 807 | 1062 |
| 28 | 87 | 116 | 118 | 262 | 560 | 670 | 956 |
| 29 | 80 | 74 | 119 | 546 | 635 | 548 | 1165 |
| 30 | 55 | 73 | 104 | 349 | 581 | 831 | 883 |
| 31 | 38 | 62 | 93 | 351 | 529 | 673 | — |
| 32 | 47 | 87 | 134 | — | 563 | 832 | — |
| 33 | 37 | 76 | 115 | — | 423 | 672 | — |
| 34 | 38 | — | 167 | — | 702 | 865 | — |
| 35 | 36 | — | 169 | — | 709 | 909 | — |
| 36 | 38 | — | 132 | — | | 891 | — |
| 37 | — | — | 147 | — | | 710 | — |

Based on the cell growth curve data in Table 2, a schematic diagram of the growth curve of the induced gastruloids is obtained as shown in B of FIG. 3. The horizontal axis of B of FIG. 3 is the induction culture time (days), and the vertical axis is the number of cells in a single gastruloid.

Referring to the growth curve data in Table 1 and the growth curve schematic diagram shown in FIG. 3, it can be seen that the cells in gastruloid proliferated steadily, from an initial cell amount of 50-75 cells on day 0 to a cell amount of 986 cells on day 7.

In summary, the cells of the induced gastruloid proliferate rapidly, have active cell growth, good cell activity, high cell induction stability, and have stable cell growth characteristics for in vitro induction.

### Example 8 Use of an In Vitro Gastruloid Induction Model Derived from Human Pluripotent Stem Cells

Thalidomide was selected for model testing of drugs that cause early embryonic teratogenesis. Two gradients of 5 μM and 10 μM were selected, and a blank control group and a dimethyl sulfoxide group (with thalidomide solvent) were added for testing.

When human induced pluripotent stem cells DYR0100 grew to 80-90% confluence, TrypLE Select was used to digest the cells into single cells, and the cells were resuspended in PBS to obtain a cell suspension 1; cell suspension 1 containing 9.0×10⁴ cells was taken, 2 mL of the G1ER culture medium containing Y-27632 was added to resuspend the cells, and then 5 μM, 10 μM of 1% % thalidomide, dimethyl sulfoxide solution and a blank control group were added respectively, a total of 4 groups, to obtain a cell suspension 2; the cell suspension 2 was inoculated into a well of an Agreewell 400™ culture dish coated with the anti-adhesion solution (Anti-adhesion rinsing Solution) in advance. After centrifugation at 800 rpm for 2 minutes using a horizontal centrifuge, the cell amount per microwell was 50-75 cells (each well of the Agreewell 400™ culture dish has 1200 microwells, that is, the cell amount in each microwell was 9.0×10⁴/1200=75 cells).

On day 2 of the culture, half of the culture medium was replaced with the G1ER culture medium, and 5 μM and 10 μM of 1% % thalidomide, dimethyl sulfoxide solution and a blank control were added respectively. Half of the culture medium was replaced every day until day 3 of the culture.

When the first stage of induction was completed, samples were taken from the four groups, and recorded with white light, then 4% paraformaldehyde was used for fixation for 30 minutes, and relevant immunofluorescence staining was performed for statistical verification.

On day 3 of the culture, 1.75 mL of the first-stage GIER culture medium was discarded, and the second-stage G2EE culture medium was added slowly along the wall, 5 μM and 10 μM of 1% thalidomide, dimethyl sulfoxide solution and a blank control were added respectively, to prevent the formed gastruloids from floating up from the microwells and then fusing.

On day 4 of the culture, 1.75 mL of the second-stage G2EE culture medium was discarded, and the third-stage aG2EE culture medium was added slowly along the wall, and 5 μM and 10 μM of 1% % thalidomide, dimethyl sulfoxide solution and a blank control were added respectively, to prevent the formed gastruloids from floating up from the microwells and then fusing.

On day 5 of the culture, 1.75 mL of the third-stage aG2EE culture medium was discarded, and the fourth-stage bG2EE culture medium was added slowly along the wall, and 5 μM and 10 μM of 1% % thalidomide, dimethyl sulfoxide solution and a blank control were added respectively, to prevent the formed gastruloids from floating up from the microwells and then fusing. After 24 hours, the culture medium was continuously replaced by 1.75 mL (the bG2EE culture medium) and cultured until day 7.

When the four-stage induction was completed, samples were taken from the four groups, and recorded the white light, then 4% paraformaldehyde was used to fix for 30 minutes, and relevant cell nucleus staining was performed for statistical verification.

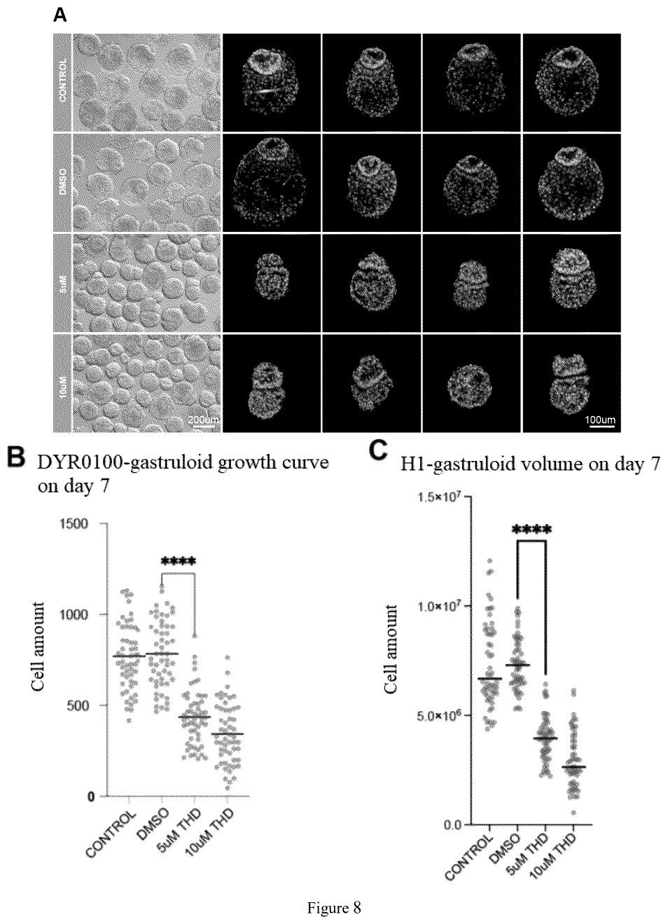

It was clearly seen from the white light results of the samples taken on day 7 that compared with the control group, the gastruloids in the dosed group had a reduced volume and the internal cavity structure was absent. By using DAPI staining to further mark the cell nucleus, it was found that compared with the control group, the internal structure of the gastruloids in the dosed group was relatively disordered, and the amniotic cavity and yolk sac could not be formed, and only germ layer separation occurred (A of FIG. 8).

At the same time, the cell amount and the volume of the single gastruloid in the control group and the dosed group were further counted and statistically analyzed. It was found that with the increase of dosed concentration, the cell amount and the volume of the gastruloid decreased, and there was a significant statistical difference compared with the blank control group and the dimethyl sulfoxide control group (B and C of FIG. 8).

It can be seen that the gastruloid model, or the tissue or organ derived from the model, or culture thereof can be used to screen, verify, evaluate, assess or study the efficacy of drugs for preventing and/or treating diseases of human early embryonic development.

Although the present disclosure has been described to a certain extent, it is obvious that appropriate changes in various conditions can be made without departing from the spirit and scope of the present disclosure. It is understood that the present disclosure is not limited to the embodiments described, but belongs to the scope of the claims, which includes equivalent replacements of each factor described. The specification and its drawings of the present disclosure are illustrative and do not constitute a limitation of the claims. The scope of protection of the present disclosure is defined by the claims and their equivalents. The specification of the present disclosure contains multiple inventive concepts, such as “preferably”, “according to a preferred embodiment” or “optionally”, which all indicate that the corresponding paragraph discloses an independent concept, and the applicant reserves the right to file a divisional application based on each inventive concept. Throughout the text, the feature guided by “preferably” is only an optional method and should not be understood as a must-have setting. Therefore, the applicant reserves the right to abandon or delete the relevant preferred features at any time.

1. A method for constructing a gastruloid model, wherein, the method comprises the following steps:
(1) digesting pluripotent stem cells into single cells when the cells grow to 80%-90% confluence, and resuspending the cells using PBS to obtain a cell suspension 1;
taking the cell suspension 1 and adding a GIER culture medium containing Y-27632 to further resuspend the cells to obtain a cell suspension 2;
inoculating the cell suspension 2 into an embryoid body culture plate coated with an anti-adhesion solution in advance, and centrifuging; and
replacing half of the culture medium with a GIER culture medium on the second day of culture, and replacing half of the culture medium every day until a primitive endoderm and a primitive ectoderm are formed;
(2) discarding ½ to ¾ of the volume of the GIER culture medium in each well of the culture plate in step (1) and adding a G2EE culture medium for culturing, and performing amniotic cavity pre-induction;
(3) discarding ½ to ¾ of the volume of the G2EE medium in each well of the culture plate in step (2) and adding an aG2EE medium for culturing, and inducing the formation of amnion cells; and
(4) discarding ½ to ¾ of the volume of the aG2EE culture medium in each well of the culture plate in step (3) and adding a bG2EE culture medium for culture until the cells are induced into amnion cells, primitive streak cells, and primordial germ cells, the post-implantation gastruloid model is obtained.

2. The method according to claim 1, wherein the pluripotent stem cells are induced pluripotent stem cells or embryonic stem cells.

3. The method according to claim 1, wherein the components of the GIER culture medium in step (1) comprise:
50% by volume of G-1™ Plus blastomere culture medium, 25% by volume of Essential 8 culture medium and 25% by volume of RACL culture medium; where
the RACL culture medium contains 95%-98.5% by volume of basic culture medium RPMI 1640 (containing GlutaMAX), 1% by volume of B27 additive, 0.5% by volume of Penicillin-Streptomycin, 0.1 mM non-essential amino acids, 0.1 mM β-mercaptoethanol, 100 ng/mL recombinant human activin A protein, 20 ng/mL recombinant human leukocyte factor protein, and 3 μM CHIR 99021; and
the components of the GIER culture medium containing Y-27632 comprise:
50% by volume of G-1™ Plus blastomere culture medium, 25% by volume of Essential 8 culture medium and 25% by volume of RACL culture medium; where
the RACL culture medium contains 95%-98.5% of basic culture medium RPMI 1640 (containing GlutaMAX), 1% by volume of B27 additive, 0.5% by volume of Penicillin-Streptomycin, 0.1 mM non-essential amino acids, 0.1 mM β-mercaptoethanol, 100 ng/ml recombinant human activin A protein, 20 ng/mL recombinant human leukocyte factor protein, 3 μM CHIR 99021, and 10 UM Y-27632.

4. The method according to claim 1, wherein the components of the G2EE culture medium in step (2) comprise:
50% by volume of G-2™ Plus embryo culture medium, 25% by volume of Essential 8 culture medium, and 25% by volume of EBB culture medium; where
the EBB culture medium contains 100% Essential 6 culture medium, 20 ng/ml recombinant human bone morphogenetic protein 4, and 10 ng/mL recombinant human fibroblast growth factor 2.

5. The method according to claim 1, wherein the components of the aG2EE culture medium in step (3) comprise:
50% by volume of G-2™ Plus embryo culture medium, 25% by volume of Essential 8 culture medium, and 25% by volume of an aEBB culture medium; where
the aEBB culture medium contains 100% Essential 6 culture medium, 100 ng/ml recombinant human bone morphogenetic protein 4, and 10 ng/mL recombinant human fibroblast growth factor 2.

6. The method according to claim 1, wherein, the components of the bG2EE culture medium in step (4) comprise:
50% by volume of G-2™ Plus embryo culture medium, 25% by volume of Essential 8 culture medium, and 25% by volume of a bEBB culture medium; where
the bEBB culture medium contains 100% Essential 6 culture medium, 75 ng/ml recombinant human bone morphogenetic protein 4, and 10 ng/mL recombinant human fibroblast growth factor 2.

7. A gastruloid model, wherein the gastruloid model is constructed by the method according to claim 1.

8. A method for studying the mechanism of human early embryonic development, comprising applying the gastruloid model according to claim 7, or tissues or organs derived from the model, or cultures thereof.

9. A method for diagnostic strategies and/or therapeutic strategies for diseases of human early embryonic development, comprising applying the gastruloid model according to claim 7, or tissues or organs derived from the model, or cultures thereof.

10. A method for screening, verifying, evaluating, assessing or studying of the efficacy of the medicament for preventing and/or treating diseases of human early embryonic development, comprising applying the gastruloid model according to claim 7, or tissues or organs derived from the model, or cultures thereof.

11. The method according to claim 1, wherein, the ratio of the cell suspension 1 to the GIER culture medium containing Y-27632 is: adding 2 mL of the GIER medium containing Y-27632 to each cell suspension 1 containing 7.5×10⁴ cells to 9.0×10⁴ cells.

12. The method according to claim 1, wherein, the cell amount of the cell suspension 2 inoculated in each microwell is 50-75 cells.

13. The method according to claim 1, wherein, step (1) is performed on day 0 to day 3 of the method.

14. The method according to claim 1, wherein, step (2) is performed on day 4 of the method.

15. The method according to claim 1, wherein, step (3) is performed on day 5 of the method.

16. The method according to claim 1, wherein, step (4) is performed on day 6 to day 7 of the method.

17. The method according to claim 2, wherein, the pluripotent stem cells are human induced pluripotent stem cells or human embryonic stem cells.

18. The method according to claim 10, wherein, the diseases of human early embryonic development are human early embryo teratogenesis caused by drugs.
